(19) United States
(12) Patent Application Publication
(10) Pub. No.: US 2014/0030212 A1
(43) Pub. Date: Jan. 30, 2014

(54) COMPOSITIONS, METHODS AND DEVICES FOR ACTIVATING AN IMMUNE RESPONSE

(71) Applicant: InCytu, Inc., Lincoln, RI (US)

(72) Inventors: Alfred V. Vasconcellos, Cranston, RI (US); William J. Bell, Hampton, CT (US); Joleen M. Medeiros, Johnston, RI (US); Jebecka Hudak, North Attleboro, MA (US); Tracie Fradet, North Attleboro, MA (US)

(21) Appl. No.: 13/917,516

(22) Filed: Jun. 13, 2013

Related U.S. Application Data
(60) Provisional application No. 61/659,355, filed on Jun. 13, 2012.

(51) Int. Cl. A61K 39/39; A61K 38/19

(52) U.S. Cl. CPC A61K 39/39; A61K 38/193
USPC 424/85.1; 424/278.1; 424/184.1

(57) ABSTRACT

The invention provides a method, system, process, vaccine, or device for activating an immune response against a tumor. In particular, in one embodiment, the invention for activating an immune response in situ against a tumor comprises introducing one or more delivery devices having a morphology that prioritizes one or more prioritized cell types which interface with the one or more delivery devices. In another embodiment, the invention provides a method of vaccinating to activate the innate immune system of a subject which comprises administering a vaccine comprising a composition selected from a group consisting of: a selection factor, an antigenic target, an immunogenic enchancing factor, and combinations thereof.

### CROSS REFERENCE TO RELATED APPLICATION

This non-provisional patent application is related to and claims priority from earlier filed U.S. Provisional Patent Application No. 61/659,355 filed Jun. 13, 2012, which is incorporated herein by reference.

### BACKGROUND OF THE INVENTION

The invention provides a composition, method, system, process, vaccine, or device for activating an immune response against a tumor. In particular, in one embodiment, the invention for activating an immune response in situ against a tumor comprises introducing one or more delivery devices having a morphology that prioritizes one or more of the prioritized cell types to interface with the one or more delivery devices. In another embodiment, the invention provides a method of vaccinating to activate the innate and the immune system of a subject which comprises administering a vaccine comprising a composition selected from a group consisting of: a selection factor, an antigenic target, an immunogenic enhancing factor, and combinations thereof.

An antigen-presenting cell (APC) is a specialized type of white blood cell (leukocyte) that helps fight off foreign substances, i.e., pathogens or infective agents that enter the body. When an APC identifies a foreign pathogen, it, in effect, acts as a sentinel by sending a signal to the immune system to create T-cells. Each type of T-cell is specially equipped to deal with different pathogens, which are typically bacteria, viruses or toxins, but can be cells in the body that the immune system fails to recognize as “self” as opposed to “non-self”. When the APC finds a pathogen, it engulfs it and enzymes inside the APC break it down into smaller particles. These processed “antigens” are then transported to the surface of the APC, and bound with either an MHC (major histocompatibility complex) class I or class II molecule. This surface presented complex forms an epitope that the T-cell can recognize and bind to via a T-cell receptor (TCR).

APCs are divided into two categories—professional and non-professional APCs. Professional APCs express MHC class II proteins and non-professional APCs express MHC class I proteins. Non-professional APCs include fibroblasts, thymic epithelial cells, thyroid epithelial cells, glial cells, pancreatic beta cells and vascular endothelial cells. There are three main types of professional APCs: macrophages, dendritic cells and B-cells. These professional APCs are able to engulf antigen quickly during a process called phagocytosis. Once the T-cell recognizes and binds to the MHC molecule complex, the APC sends out an additional co-stimulatory signal to activate the T-cell. Professional APCs are able to activate helper T-cells that have never encountered their antigens.

Macrophages are white blood cells that are ubiquitously located in vertebrate tissues. They originate from monocytes in the bone marrow. Upon activation, they travel to the site of injury, and they engulf and digest antigens through phagocytosis.

B-cells produce antibodies (immunoglobulin) that are specific to certain antigens. B-cells are able to efficiently present the antigen to which their antibody is directed, but they are considered inefficient APCs for most other antigens. B-cells are continually produced in the bone marrow. Immature B-cells only express IgM (immunoglobulin type M) on their cell membrane. Once the B-cell reaches maturity, it can express both IgM and IgD (immunoglobulin type D) on the cell surface. This mature cell is now able of responding to antigens. Once the immunoglobulin molecule interacts with an antigen, the B-cell becomes activated and differentiates into many antibody-producing cells (plasma cells). Each plasma cell secretes millions of identical antibody molecules, which are released into the bloodstream. Some of the plasma cells will undergo isotype switching, during which the cell expresses other immunoglobulin isotypes, including IgA, IgE and IgG.

Dendritic cells (DCs) constitute only about 0.3% of all circulating blood leukocytes. They are mainly present in the skin (where they are called langerhans cells) and the inner lining of the nose, stomach, lungs and intestines because these locations are the primary entrances to foreign pathogens. Immature dendritic cells (also called veiled cells) are found in the bloodstream and their pattern recognition receptors (PRRs) are constantly sampling their surroundings for pathogens, such as bacteria and viruses or other foreign substances (i.e, antigens). For this reason, DCs are potent activators in the immune system. Upon encountering an antigen, the DCs process the antigen by forming a Major Histocompatability Complex (MHC) with the antigen on the cell's surface. This process, or MHC pathway, and the resultant MHC-peptide complex is capable of stimulating CD4+ type T cells. DCs also possess a unique ability to “cross-present” antigens; DC endosomes release captured antigenic material into the cytosol where it is broken down by proteasomes. The degraded peptides are then transported to the endoplasmic reticulum via a transporter-associated protein (TAP) and bound to MHC-class I molecules for presentation to CD8+ T cells. By these separate mechanisms, DCs stimulate in both a MHC-class I and MHC class II manner, and diversify the immune response to an antigen.

Once APCs have engulfed the antigen, chemokines attract the APCs and assist in their migration to the lymph nodes where most T-cells are located. Chemokines are chemical mediators in the blood that are produced by cytokines. During the migration, the APC cells lose much of their ability to engulf antigens and develop an increased ability to communicate with T-cells. Once the antigenic epitopes are combined with MHC and presented to the T-cell surface, helper T-cells activate the APCs to produce antibodies against the antigen.

One cytokine, Granulocyte Macrophage Colony Stimulating Factor (GM-CSF), is also present in increased concentrations during inflammation. GM-CSF is a hematopoietic (multi-lineage) growth factor. It stimulates stem cells to produce neutrophils, eosinophils, and basophils (granulocytes) and to produce monocytes. GM-CSF also inhibits neutrophil migration. Monocytes leave the circulatory system and migrate into tissue, where they mature into macrophages and DCs. Thus, GM-CSF and its presence cause both the recruitment of additional monocytes and DCs to the inflammation site and the induction of local monocytes to transform into DCs. The active form of GM-CSF is found extracellularly as a homodimer. Sieff et al., “Human recombinant granulocyte-macrophage colony-stimulating factor: a multilineage hematopoietin”, Science 230: 1171-73 (1985).

Although there is a broad array of bioactive GM-CSFs, one of the products with the most significant clinical history is sargramostim (marketed in the United States as LEUKINE™). Sargramostim is a recombinant human granulocyte macrophage colony stimulating factor (rhu GM-CSF) produced by recombinant DNA technology in a yeast (S. cerevisiae) expression system. Sargramostim is used clinically to stimulate proliferation and differentiation of hematopoietic progenitor cells. It is a glycoprotein of 127 amino acids characterized by three primary molecular species having molecular masses of 19,500, 16,800 and 15,500 daltons. The amino acid sequence of sargramostim differs from the natural human GM-CSF by a substitution of leucine at position 23, and the carbohydrate moiety may be different from the native protein.

T cells originate from hematopoietic stem cells in the bone marrow. Progenitor cell lines derived from these stem cells populate the thymus and expand by cell division to generate a large population of immature thymocytes, which express neither CD4 nor CD8 proteins, and are therefore classed as double-negative (CD4−CD8−) cells. As they progress through their development they become double-positive thymocytes (CD4+ CD8+), and finally mature to single-positive (CD4+ CD8− or CD4−CD8+) thymocytes that are then released from the thymus to peripheral tissues. CD4+ CD8− (or simply CD4+) cells are T helper cells (TH cells) that assist other white blood cells in immunologic processes, including maturation of B cells and activation of cytotoxic T cells and macrophages. Cytotoxic T cells (TC cells, or CTLs) destroy virally infected cells and tumor cells, and are also implicated in transplant rejection. These cells are also known as CD8+ T cells. Regulatory T cells (Treg cells) are essential in the maintenance of immunological tolerance. Their primary role is to terminate T cell-mediated immunity toward the end of an immune reaction and to suppress auto-reactive T cells that escaped the process of negative selection in the thymus. Two major classes of CD4+ regulatory T cells have been described, including the naturally occurring Treg cells and the adaptive Treg cells. Naturally occurring Treg cells (also known as CD4+ CD25+FoxP3+Treg cells) are involved in interactions between developing T cells and activated dendritic cells. Adaptive Treg cells (also known as Tr1 cells or Th3 cells) may originate during a normal immune response.

Therapeutic vaccines based on DCs carrying tumor antigens have emerged as a strategy to initiate an immune response against tumor cells. These vaccines can be prepared using different methodologies, such as the application of tumor mRNA (see for example Sousa-Canavez et al, “Therapeutic dendritic cell vaccine preparation using tumor RNA transfection: A promising approach for the treatment of prostrate cancer”, Genetic Vaccines and Therapy 6: 2 (2008).

Other antigen sources that have been employed include synthetic peptides (see for example Prins et al, “Immunotherapeutic targeting of shared melanoma-associated antigens in a murine glioma model”, Cancer Res. 63: 8487-91 (2003)), acid-eluted tumor peptides (see for example Liau et al, “Treatment of intracranial gliomas with bone marrow-derived dendritic cells pulsed with tumor antigens, J. Neurosurg. 90: 1115-24 (1999)), tumor lysate (see for example Grauer et al., “Tumor lysate-pulsed dendritic cells in a murine glioma model”, Int J Cancer 122: 1794-1802 (2008)), DC-tumor fusion cells (see for example, Akasaki et al, “Antitumor Effect of Immunizations with Fusions of Dendritic and Glioma Cells in a Mouse Brain Tumor Model”, J Immunotherapy 24: 106-113 (2001)), antigen containing vectors (see for example Yamanaka et al, “Administration of interleukin-12 and -18 enhancing the antitumor immunity of genetically modified dendritic cells that had been pulsed with Semliki forest virus-mediated tumor complementary DNA”, J Neurosurg. 97: 1184-90 (2002)), and tumor extract carrying liposomes (see for example, Aoki et al., “Dendritic cells pulsed with tumor extract-cationic liposome complex increase the induction of cytotoxic T lymphocytes in mouse brain tumor”, Cancer Immunol Immunotherapy 50: 463-468 (2001)). These types of tumor vaccines have shown some results for treating brain tumors. See for example Yu et al., “Vaccination of malignant glioma patients with peptide-pulsed dendritic cells elicits systemic cytotoxicity and intracranial T-cell infiltration”, Cancer Res. 61: 842-87 (2001) and Kim and Liau, “Dendritic cell vaccines for brain tumors”, Neurosurg Clin N Am 21 (1): 139-57 (2010).

The use of cytokines to supplement DC-based therapy is detailed in Kim, et al, “Enhancement of antitumor immunity of dendritic cells pulsed with heat-treated tumor lysate in murine pancreatic cancer”, Immunol Lett. 103: 142-48 (2006); Akasaki et al, supra; Yamanaka et al, supra; and Kikuchi et al, “Vaccination of glioma patients with fusions of dendritic and glioma cells and recombinant human interleukin 12”, J Immunol. 162: 168-75 (2004). International Patent Application No. PCT/US2009/000914, published as WO 2009/102465, discloses compositions and implantable scaffolds for stimulating an anti-tumor immune response. Briefly, that document discloses implants composed of macroporous PLG (poly[lactide-co-glycolide]) scaffolds that incorporate tumor antigen/lysate and other molecules such as encapsulated GM-CSF (in order to attract dendritic cells to the scaffold) and CpG-ODN (Cytosine-guanosine oligonucleotide, which is known to stimulate DC activation). The scaffolds are implanted near the tumor site. The device is said to temporally control local GM-CSF concentration by releasing a certain amount of GM-CSF in a pulse fashion within 1-7 days of implantation, following which the residual amount of GM-CSF is released slowly over an extended period of time, for example from 1-12 days or 2-5 or more weeks.

Therapeutic vaccines based on DCs carrying tumor antigens have emerged as a strategy to initiate an immune response against tumor cells. However, current approaches are far from optimal in that many patients treated with DC vaccines have failed to respond. Moreover, ex vivo manipulation of DCs is time consuming and costly. Therefore, there is a desire to improve the existing methods above and provide a method for activating in situ an immune response against a tumor in a subject in a more effective manner.

### BRIEF SUMMARY OF THE INVENTION

The invention provides a composition, method, system, process, vaccine, or device for activating an immune response against a tumor. In particular, in one embodiment, the invention for activating an immune response in situ against a tumor comprises introducing one or more delivery devices having a morphology that prioritizes one or more prioritized cell types to interface with the one or more delivery devices. In another embodiment, the invention provides a method of vaccinating to activate the innate and adaptive immune system of a subject which comprises administering a vaccine comprising a composition selected from a group consisting of: a selection factor, an antigenic target, an immunogenic enhancing factor, and combinations thereof.

And in a further embodiment, the invention provides a method of vaccinating to activate the innate and adaptive immune system of a subject which comprises administering a vaccine comprising a composition selected from a group consisting of: a selection factor, an antigenic target, an immunogenic enhancing factor, and combinations thereof so that a long term immunological memory is created.

The method of producing an immune response in situ of a subject comprises introducing one or more delivery devices having a morphology that prioritizes one or more prioritized cell types which interface with the one or more delivery devices. The prioritized cell types are selected from a group consisting of: NK cells, innate T cells, dendritic cells, other antigen presenting cells, and combinations thereof.

The one or more delivery devices comprises a composition selected from a group consisting of: a selection factor, an antigenic target, and combinations thereof. In another embodiment, the composition may further comprise an immunogenic enhancing factor.

The selection factor of the composition is configured to prioritize the prioritized cell types which interface with the one or more delivery devices. In particular, the selection factor is configured to actively attract, proliferate, or mature antigen presenting cells. For example, the selection factor is a cytokine. More specifically, by way of example, of the selection factor may be GM-CSF. Of course, the selection factor is not limited to GM-CSF and may include other cytokines. Other types of selection factors may also be used which can prioritize or increase the presence or receptivity of the prioritized cell types which improves the interface with one or more delivery devices. The selection factor increases the probability of interaction of the prioritized cell types with one or more delivery devices when compared to non-prioritized cell types.

The antigenic target of the composition may include, without limitation, tumor lysates extracted from biopsies, irradiated tumor cells, tumor cells, MAGE antigens, MART-1/melana, tyrosinase, ganglioside, gp-100, GD-2, GM-2, O-acetylated GD-3, MUC-1, Sos1, Protein kinase C-binding protein, reverse transcriptase protein, AKAP protein VRK1, Kiaa1735, T7-1, T11-3, T11-9, Homo Sapiens telomerase ferment (HRTR), cytokeratin-19, Squamous cell carcinoma antigens 1 and 2, ovarian carcinoma antigens, carcinoma associated mucins, CTCL tumor antigens, prostate specific membrane antigens, 5T4 oncofetal trophoblast glycoprotein, Orf73, colon cancer antigen NY-CO-45, lung cancer antigen NY-LU-12, cancer associated surface antigen, adenocarcinoma antigen ART1, paraneoplastic associated brain testis cancer antigen, NOVA2, hepatocellular carcinoma antigen, tumor-associated antigens, breast cancer antigens NY-BR-15 and -16, chromogranin A, parathyroid secretory protein 1, DUPAN-2, CA 19-9, 72-4 and 195, and CEA.

The immunogenic enhancing factor of the composition may include, without limitation, CpG-ODN. Other CpG sequences or derivatives are also known in the art and may be employed in place of CpG-ODN. Exemplary are ODN 1585, ODN 1668, ODN 1826, ODN 2006, ODN 2006-G5, ODN 2216, ODN 2336, ODN 2395, ODN M362, each of which may be obtained from InvivoGen (San Diego, Calif.). In another embodiment, a liposome contains the immunogenic enhancing factor. Also, the immunogenic enhancing factor may include an adjuvant. In addition, other immunogenic enhancing factors that are known in the art may be used.

The one or more delivery devices may be introduced in a variety of configurations. In one embodiment, a first delivery device releases the selection factor and a second delivery device releases the antigenic target and the immunogenic enhancing factor. In another embodiment, a second delivery device is located proximally to the first delivery device within 0 to 28 days after introducing the first delivery device. In a more preferred range, the second delivery device is located proximally to the first delivery device within 1 to 10 days after introducing the first delivery device.

In another embodiment, the one or more delivery devices comprising at least the antigenic target are located proximal to a tumor, and one or more of the delivery devices continuously releases the selection factor to facilitate acquisition of the antigenic target by antigen presenting cells or other prioritized cell types.

In another embodiment, the one or more delivery devices comprising at least the antigenic target are located proximal to a tumor, and one or more of the delivery devices continuously releases the antigenic target to facilitate acquisition of the antigenic target by antigen presenting cells.

In another embodiment, the one or more delivery devices comprising at least the antigenic target are located proximal to a lymph node, and one or more of the delivery devices continuously releases the antigenic target to facilitate acquisition of the antigenic target by antigen presenting cells or other prioritized cell types.

The one or more delivery devices are configured to have a size and shape to facilitate antigen presenting cells acquisition of the antigenic target. The one or more delivery devices are configured to have surface pores whose size and shape facilitate the acquisition of the antigenic target from the one or more delivery devices by antigen presenting cells or other prioritized cell types. Such morphology varies depending on the targeted prioritized cell type

In addition, the one or more delivery devices may comprise one or more bioresorbable delivery devices. In one embodiment, the one or more bioresorbable delivery devices are configured to present the antigenic target on the surface of the one or more bioresorbable delivery devices which are renewed when the one or more bioresorbable delivery devices resorbs in vivo to provide fresh antigen for the antigen presenting cells. In another embodiment, the one or more bioresorbable delivery devices are configured to locally release the antigenic target as the one or more bioresorbable delivery device resorbs to provide a locally increased concentration of the antigenic target for the antigen presenting cells to acquire. Further, the one or more bioresorbable delivery device releases small particles to facilitate the antigen presenting cells acquisition of the antigenic target.

In another embodiment, an injection is administered comprising the immunogenic enhancing factor or the immunogenic factor and the antigenic target at an area proximal to the location of the one or more delivery devices. In another embodiment, the immunogenic enhancing factor and the antigenic target are transdermally administered at an area proximal to the location of the one or more delivery devices.

In another embodiment, the composition is released in a bimodal manner with a first, initial burst of the composition being released within a first time period of 72 hours or less, and immediately following thereafter, a second, more gradual release of the composition continuing for a second time period. In one embodiment, 90% or less of the total content of the composition is released in vivo in the initial burst within the first time period. In another embodiment, between 1 and 10% of the total content of the composition is released in vivo in the initial burst within the first time period.

A remaining content of the composition is released within the second time period and at a rate no greater than 1% of the content per each 24 hour period. In another embodiment, the rate is no greater than 0.10% of the remaining content per each 24 hour period over a period of at least three weeks. After three weeks, the remaining content releases per each 24 hour period which varies between 0% and 35% of the remaining volume of composition.

In another embodiment of the bimodal release, the composition is released in a bimodal manner with an initial burst of between about 50% and about 60% of the content of the composition released in a pulse within a first time period of 24 hours or less after implantation, and immediately following thereafter, a second, more gradual release of the composition continuing for a second time period. In one embodiment, the first time period is 1 to 7 days after implantation.

In an alternative embodiment, a bolus comprising a selection factor is administered and the one or more delivery devices implanted comprise an immunogenic enhancing factor and antigenic target. In one embodiment, for the selection factor to assist in the recruitment of DCs, the selection factor must be administered in a single spike, either released upon implantation of the device or separately administered, for example as a bolus injection. Unless the DCs are attracted to the presentation surfaces of the device before other immune system cells coat the device in a foreign body response, the ability of the DCs to take up antigenic target is greatly diminished.

The composition may be released in a biologically appropriate dosage density and flux which does not attract unwanted cell types due to triggering an inflammatory or necrotic response. For example, but in no way limited to, the biologically appropriate dosage and flux of the selection factor is between 0.1 and 600 nanograms/mm²/day. In another example, but in no way limited to, the biologically appropriate dosage and flux of the antigenic target is between 0.1 and 600 nanograms/mm²/day. In another example, the biologically appropriate dosage and flux of the immunogenic enhancing factor is between 0.1 and 600 nanograms/mm²/day. Of course, the biologically appropriate dosage and flux of the composition may be amended or changed depending upon the need to not attract unwanted cell types which may result in triggering an inflammatory or necrotic response.

The method of vaccinating to activate the innate immune system of a subject comprises administering a vaccine comprising a composition selected from a group consisting of: a selection factor, an antigenic target, and combinations thereof. The composition may further include an immunogenic enhancing factor. Upon vaccination, in one embodiment, the innate immune system is activated within 24 hours of vaccination. The selection factor may be configured to minimize the controlling effect of monocytes on the NK cells. The selection factor is also configured to attract, proliferate, or mature antigen presenting cells.

In general, the composition of the vaccine may be configured in a variety of ways. The composition may be configured to activate antigen presenting cells to initiate and maintain an immune response. The composition may also be configured to maintain the presentation of the antigenic target and selection factor for a defined time period to facilitate a memory immune response which outlasts the presentation of the antigenic target. The composition may also be configured to effect the adaptive immune system to minimize the upregulation of regulatory cells.

### BRIEF DESCRIPTION OF THE DRAWINGS

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 shows an example of how DCs are activated upon implantation of a delivery device or vaccination.

### DETAILED DESCRIPTION OF THE PREFERRED EMBODIMENTS

The invention provides a composition, method, system, process, vaccine, or device for activating an immune response against a tumor. In particular, in one embodiment, the invention for activating an immune response in situ against a tumor comprises introducing one or more delivery devices having a morphology that prioritizes one or more prioritized cell types which interface with the one or more delivery devices. In another embodiment, the invention provides a method of vaccinating to activate the innate and adaptive immune system of a subject which comprises administering a vaccine comprising a composition selected from a group consisting of: a selection factor, an antigenic target, an immunogenic enhancing factor, and combinations thereof. In one embodiment, the invention is used to develop a cancer vaccine.

The method of producing an immune response in situ of a subject comprises introducing one or more delivery devices having a morphology that prioritizes one or more prioritized cell types which interface with the one or more delivery devices. More particularly, the method of activating in situ an immune response against a tumor in a mammalian subject includes implanting, locating, or other methods of introduction at the desired site in the body of the subject the one or more delivery devices. It should be noted that the desired site may be located in the lymph node area. Without limitation, the one or more delivery devices or related compositions may be put inside, encompassed, included, inserted, transplanted, or otherwise into the subject of the body in one or more locations. The one or more delivery devices may be bioactive and introduced subcutaneously.

The prioritized cell types are selected from a group consisting of: NK cells, innate T cells, dendritic cells, other antigen presenting cells, and combinations thereof. For example, in one embodiment, the prioritized cell type is a dendritic cell.

The one or more delivery devices comprises a composition selected from a group consisting of: a selection factor, an antigenic target, and combinations thereof. In another embodiment, the composition may further comprise an immunogenic enhancing factor. It should be appreciated the composition may be provided in a variety of forms, volumes, configurations, combinations with other materials, and delivered with or without delivery devices or other methods for delivering the composition into the subject.

The selection factor of the composition is configured to prioritize the prioritized cell types which interface with the one or more delivery devices. In particular, the selection factor is configured to actively attract, proliferate, or mature antigen presenting cells. For example, the selection factor is a cytokine. More specifically, by way of example, of the selection factor may be GM-CSF. Of course, the selection factor is not limited to GM-CSF and may include other cytokines. Other types of selection factors may also be used which can prioritize or increase the presence or receptivity of the prioritized cell types which improves the interface with one or more delivery devices. The selection factor increases the probability of interaction of the prioritized cell types with one or more delivery devices when compared to non-prioritized cell types

Although the use of GM-CSF is preferred due to its history of clinical use, other cytokines and growth factors having similar capabilities such as, for example, interleukins. The GM-CSF may be glycosylated or glycosylated. The fully glycosylated form is more active in vivo and for that reason it is preferred. Other types of selection factors may also be used which can prioritize the prioritized cell types which interface with the one or more delivery devices.

Although the attraction of the dendritic cells to an antigenic target and immunogenic enhancing factor or danger signal, i.e. adjuvant, can be done from one location, the negative effects of preventing the matured dendritic cells from migrating to the lymph nodes may be avoided by having a selection factor near but not at the same location as the source of antigenic target. Accordingly, in one embodiment a preparatory source of a selection factor in a controlled release device is implanted in the patient near the lymph nodes to attract and proliferate APCs and an implant of a second device containing the immunogenic enhancing factor, the antigenic target, and the selection factor is implanted nearby.

The selection factor comprising GM-CSF may also be delivered appropriately by administering to the mammalian subject a vector having a sequence encoding GM-CSF along with the appropriate regulatory sequences in order to produce the additional required and desired amount of GM-CSF. Such vectors and required sequences are well known in the art.

In the methods of the invention, selection factor is multifunctional. First, as discussed, the selection factor is an attractant for APCs. The greater the number of APCs attracted to the delivery device surfaces, the more antigenic target is taken up and presented to the immune system. There has been a significant demonstration in traditional vaccines that this event improves therapeutic outcomes.

Second, selection factor is a differentiation and proliferative agent for DCs. This results in the same outcome as the outcome above but through a different mechanism. The number of DCs and the percentage of DCs at the surface of the delivery device can be increased by the local differentiation and proliferation APC progenitors.

Third, the selection factor inhibits APC migration to lymph nodes. Although this is a problem when it is desirable for APCs to migrate to lymph nodes, for a short period of time this is a desirable effect. This local “short term delay”, in effect, increases the resident time during which the DCs attracted to the delivery device spend in proximity to the antigenic target, thereby increasing the number of cells that pickup antigenic target and the amount of antigenic target that is picked up by the APCs. Because extended delivery of high levels of GM-CSF has been demonstrated to promote neoplastic transformation very long-term administration is contraindicated. Mann et al, ‘Up- and Down-Regulation of Granulocyte/Macrophage-Colony Stimulating Factor Activity in Murine Skin Increase Susceptibility to Skin Carcinogenesis by Independent Mechanisms”, Cancer Res 61: 2311 (2001).

Antigenic targets of the composition able to provide protective or therapeutic immunity to a subject are known in the art. Exemplary antigenic targets encompassed by the methods and devices of the invention include, without limitation, tumor lysates extracted from biopsies, irradiated tumor cells, MAGE antigens, MART-1/melana, tyrosinase, ganglioside, gp-100, GD-2, GM-2, O-acetylated GD-3, MUC-1, Sos1, Protein kinase C-binding protein, reverse transcriptase protein, AKAP protein VRK1, Kiaa1735, T7-1, T11-3, T11-9, Homo Sapiens telomerase ferment (HRTR), cytokeratin-19, Squamous cell carcinoma antigens 1 and 2, ovarian carcinoma antigens, carcinoma associated mucins, CTCL tumor antigens, prostate specific membrane antigens, 5T4 oncofetal trophoblast glycoprotein, Orf73, colon cancer antigen NY-CO-45, lung cancer antigen NY-LU-12, cancer associated surface antigen, adenocarcinoma antigen ART1, paraneoplastic associated brain testis cancer antigen, NOVA2, hepatocellular carcinoma antigen, tumor-associated antigens, breast cancer antigens NY-BR-15 and -16, chromogranin A, parathyroid secretory protein 1, DUPAN-2, CA 19-9, 72-4 and 195, and CEA.

The antigenic target employed will depend on the type of tumor to be treated. It can be a product of a mutated oncogene or tumor suppressor gene, such as for example ras or p53. It can be an overexpressed or aberrantly expressed cellular protein. Tyrosinase is an example of the former. It can be produce by a oncogenic virus such as Epstein-Barr Virus or Pappioma Virus, or an oncofetal antigen such as alphafetoprotein or carcinoembryonic antigen. Cell surface glycolipids and glycoproteins having an abnormal structure may also be employed as antigenic targets.

The immunogenic enhancing factor of the composition may include, without limitation, CpG-ODN, or other adjuvants. Other CpG sequences or derivatives are also known in the art and may be employed in place of CpG-ODN. Exemplary are ODN 1585, ODN 1668, ODN 1826, ODN 2006, ODN 2006-G5, ODN 2216, ODN 2336, ODN 2395, ODN M362, each of which may be obtained from InvivoGen (San Diego, Calif.). Other adjuvants that may be used include GM-CSF. In addition, other immunogenic enhancing factors that are known in the art may be used.

GM-CSF, CpG-ODN, and other bioactive molecules useful in decreasing or eliminating tumor burden may be administered in any of the known devices composed of biocompatible, biodegradable, polymer matrices or scaffolds. Hydrogels are exemplary, and may be formed from polylactic acid, polyglycolic acid, PLGA polymers, alginates and alginate derivatives, gelatin, collagen, agarose, natural and synthetic polysaccharides, polyamino acids, polyesters such as polyhydrosxybutyrate and poly-epsilon-caprltaction, polyanhydrides, polyphosphazines, poly(vinyl alcohols), poly(alkylene oxides) such as poly(ethylene oxides), poly(allylamines), poly(acrylates) and the like. An exemplary matrix uses an alginate or other polysaccharide of relatively low molecular weight, the size of which after dissolution is at the renal threshold for clearance by humans: between about 1000 to 80,000 daltons. It is also useful to use an alginate of high guluronate content as the guluronate units provide sites for ionic crosslinking. U.S. Pat. No. 6,642,363 discloses polymers that are particularly useful in the invention.

The one or more delivery devices may be introduced in a variety of configurations including variety of compositions, positions within the body, multiple devices, timing of introduction of one or more devices into subject, and use in conjunction with other methods of introducing the compositions into the subject. Of course, it is contemplated that multiple delivery devices with one or more features may be introduced into the subject.

Structural material of the one or more delivery devices, in one embodiment, may include a non-biodegradable material, such as metal, plastic, polymer, or silk polymer. The bioactive compositions themselves are composed of a biocompatible material which may be non-toxic or non-immunogenic. The bioactive compositions may be covalently or non-covalently to the structural material.

In one embodiment, a first delivery device releases the selection factor and a second delivery device releases the antigenic target and the immunogenic enhancing factor. In another aspect, the method includes implanting a first delivery device that includes a selection factor and a second device comprising tumor antigen which is implanted proximally that engages antigen presenting cells. In a another embodiment, a first delivery devices includes a selection factor, such as GM-CSF and an antigenic target, and a second delivery device contains a bolus of sufficient amount of the selection factor to attract or proliferate DCs to the first delivery device whose selection factor is small enough to not inhibit neutrophil migration. This balance is enabled by delivery systems with a short term bolus followed by a trace release, or by multiple injections of the selection factor of varying concentrations.

In another embodiment, two or more delivery devices are introduced into the subject at different time periods. For example, a second delivery device is located proximally to the first delivery device within 0 to 28 days after introducing the first delivery device. In a more preferred range, the second delivery device is located proximally to the first delivery device within 1 to 10 days after introducing the first delivery device.

In another embodiment, the one or more delivery devices are located in different positions or locations within the subject. For example, the one or more delivery devices comprising at least the antigenic target are located proximal to a tumor, and the one or more delivery device continuously releases the antigenic target to facilitate acquisition of the antigenic target by antigen presenting cells or other prioritized cell types. The one or more delivery device continuously presents the antigenic target to facilitate acquisition of the antigenic by an antigen presenting cells over a time period greater than 10 days.

The one or more delivery devices are configured to have a size and shape to facilitate antigen presenting cells acquisition of the antigenic target. The one or more delivery devices are configured to have surface pores whose size and shape facilitate the acquisition of the antigenic target from the one or more delivery devices by antigen presenting cells. The one or more delivery devices may define a matrix which is porous or non-porous. If porous, the diameter of the pores may range from the nanoscale having a diameter less than about 10 nm, microporous having a diameter in the range of about 100 nm-20 micrometers, or macroporous having a diameter of greater than about 20 micrometers, preferably greater than about 100 micrometers and more preferably greater than about 400 micrometers. The preparation of polymer matrices having the appropriate pore size is described in International Patent Publication WO 2009/102465 and in U.S. Pat. No. 6,511,650.

The bioactive compounds incorporated into the matricies or delivery devices may be purified naturally-occurring compounds, synthetically produced compounds, or recombinant compounds, for example, polypeptides, nucleic acids, small molecules or other anti-tumor agents. The release profile of the bioactive compounds may be controlled using different techniques, for example encapsulation, the nature of the attachment or association with the matrix, the porosity and the particle size. Such techniques, and matrix constructions, are addressed in International Patent Publication WO 2009/102465 and are known in the art.

The compounds are purified, i.e., at least 90% by weight of the compound of interest, most preferably at least 99% by weight of the compound of interest. Purity can be measured by any appropriate stand method. Coupling the compounds to the matrix may be accomplished by any method known to one of ordinary skill in the art. See for example, Hirano and Mooney, Advanced Materials, pages 17-25 (2004) and Hermanson, Bioconjugate Techniques, pages 152-185 (1996).

The device is formed so as to continuously present the antigenic target on the device surface. In addition, the delivery device has a finite surface area. Direct access to the surface of the device presents the antigenic target in a manner that is optimal for the DCs to take up the antigenic target. Because foreign substances attract a large number different types of immune system cells, the selection factor should be employed to increase the percentage of the cells that are the preferred or prioritized cell types, more specifically DCs which are at the site of the delivery device early and are able to find their way to the surface of the device and thereby access to the antigenic target is increased. Such prioritization preferentially activates the portions of the immune response that is antitumorgenic.

In addition, the one or more delivery devices may comprise one or more bioresorbable delivery devices. In one embodiment, the one or more bioresorbable delivery devices are configured to present the antigenic target on the surface of the one or more bioresorbable delivery devices which are renewed when the one or more bioresorbable delivery devices resorbs in vivo to provide fresh antigen for the antigen presenting cells. In another embodiment, the one or more bioresorbable delivery devices are configured to locally release the antigenic target as the one or more bioresorbable delivery device resorbs to provide a locally increased concentration of the antigenic target for the antigen presenting cells to acquire. Further, the one or more bioresorbable delivery device releases small particles to facilitate the antigen presenting cells acquisition of the antigenic target. The bioresorbable delivery device may be a bioresorbable polymer disk having a selection factor and antigenic target that is tumor specific presented to a localized environment in situ.

Another embodiment of the bioresorbable delivery device comprised of a bioresorbable material which is highly biocompatible and whose surface has texture in the 0.01 to 25 micron range so as to promote the close proximity and physical closeness of dendritic cells; and which releases selection factor for a period of between 2 and 36 hours prior to making available to such dendritic cells the antigenic target and/or a immunogenic enhancing factor or adjuvant such as CpG to the DCs. In another embodiment, the surface presentation of the bioresorable device is replaced by controlled release of particles specifically sized to optimize DC uptake of antigenic target and immunogenic enhancing factor (CpG).

In another embodiment, an injection is administered comprising the immunogenic enhancing factor and/or the antigenic target at an area proximal to the location of the one or more delivery devices. In another embodiment, the immunogenic enhancing factor and the antigenic target are transdermally administered at an area proximal to the location of the one or more delivery devices.

In another embodiment, the composition is released in a bimodal manner with a first, initial burst of the composition being released within a first time period of 72 hours or less, and immediately following thereafter, a second, more gradual release of the composition continuing for a second time period. A remaining volume of the composition is released within the second time period and at a rate no greater than 1% of the volume per each 24 hour period. After three weeks, the remaining volume releases per each 24 hour period which varies between 0% and 35% of the remaining volume of composition. In addition, the method may further include a third time period for the remaining composition to engage the antigen presenting cells.

In an alternative embodiment, a bolus comprising a selection factor is administered and the one or more delivery devices comprise an immunogenic enhancing factor and antigenic target. In any of these alternatively, the volume of the bolus composition administered is between 0.5 micrograms and 10 micrograms, more preferably between 2 and 4 micrograms per vaccination site.

The bolus composition may be administered at various times. The bolus composition may be administered at the time the device is implanted, within 24 hours of implantation, or within 14 days, more preferably between 3 to 5 days, prior to vaccination.

The one or more delivery device and the bolus may also be administered proximal to the tumor. The one or more delivery device and the bolus may be administered proximal to a lymph node that has at least a portion not occupied by the tumor. In the case of the lymph nodes, the device should be implanted and the bolus should be administered proximal to any portion of the lymph node not infected by the tumor.

The composition may be released in a biologically appropriate dosage density and flux which does not attract unwanted cell types due to triggering an inflammatory or necrotic response. Equally important is the density of release or amount of composition per unit of surface area. With a range of delivery between in the first 24 hours of between 0.1 and 600 nanograms/mm²/day. For example, but in no way limited to, the biologically appropriate dosage and flux of the selection factor is between 0.1 and 600 nanograms/mm²/day. In another example, but in no way limited to, the biologically appropriate dosage and flux of the antigenic target is between 0.1 and 600 nanograms/mm²/day. In another example, the biologically appropriate dosage and flux of the immunogenic enhancing factor is between 0.1 and 600 nanograms/mm²/day. Of course, the biologically appropriate dosage and flux of the composition may be amended or changed depending upon the need to not attract unwanted cell types due to triggering an inflammatory or necrotic response.

In one embodiment, the method of activating the immune response includes activating APCs, more specifically, dendritic cells in situ in a mammalian subject having a tumor. This method of the invention finds particular use in the treatment of lymph node tumors. For example, one or more delivery devices comprising an antigenic target are implanted at, on, near, or proximal to a tumor area in the body. The one or more delivery devices continuously releases antigenic target to facilitate acquisition of the antigenic target by antigen presenting cells or other prioritized cell types. The device may further include the selection factor which actively attracts, proliferates or matures the APCs or dendritic cells. The device is formed so as to continuously present the antigenic target on the device surface in order to facilitate acquisition of the antigenic target by antigen presenting cells over a time period greater than 10 days.

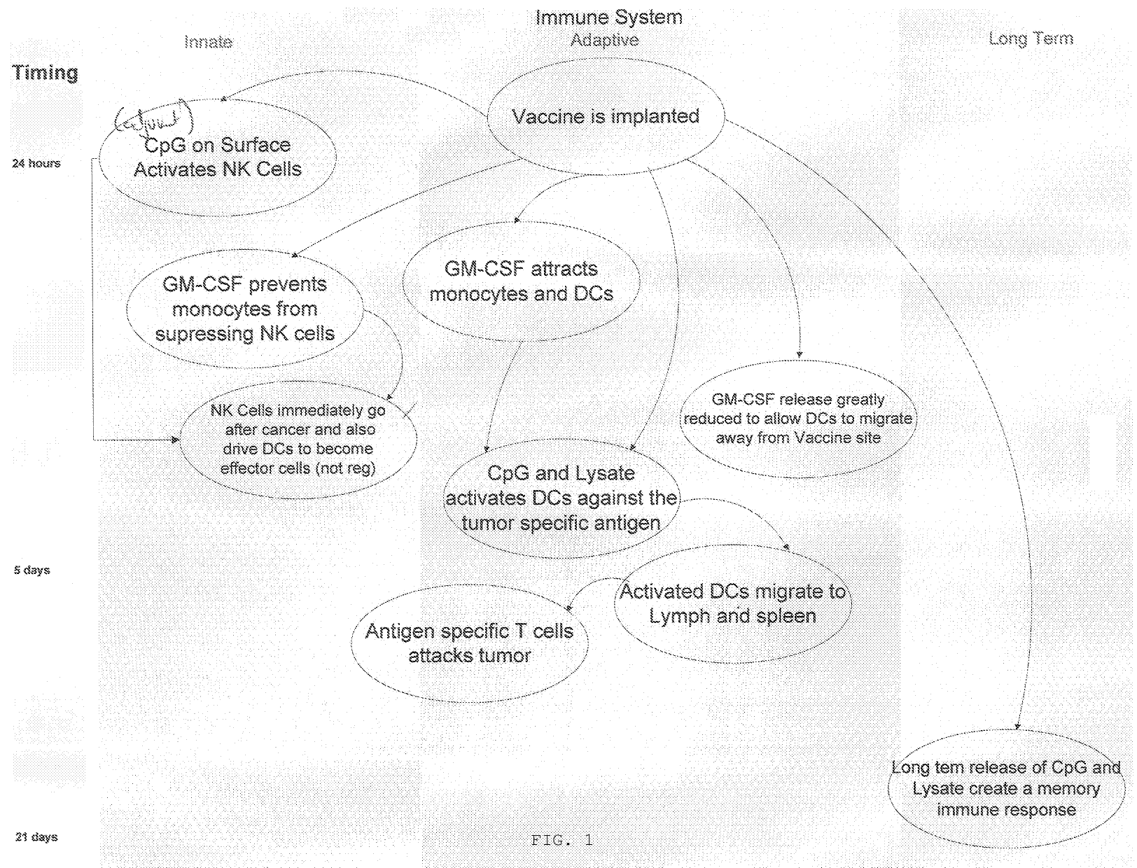

Referring to FIG. 1, an illustration of another embodiment shows how DCs are activated upon implantation of a device or vaccination. Within 24 hours of vaccination, the immunogenic enhancing factor, for example, CpG-ODN, activates NK cells. Since a selection factor, for example GM-CSF, is also administered, monocytes and DCs are attracted to the implantation or vaccination site and the selection factor prevents the monocytes from suppressing NK cells. The NK cells attack the tumor and drive DCs to become effector cells. At the same time, the immunogenic enhancing factor and a lysate activate DCs against the tumor specific antigen included in the device or vaccination. The selection factor release is greatly reduced to allow DCs to migrate away from the vaccination site. The activated DCs migrate to the lymph and spleen where antigen specific T cells are produced and attack the tumor. Long term release of the immunogenic enhancing factor and lysate or antigenic target creates a memory immune response.

The method of vaccinating to activate the innate immune system of a subject comprises administering a vaccine comprising a composition selected from a group consisting of: a selection factor, an antigenic target, and combinations thereof. The composition may further include an immunogenic enhancing factor. Upon vaccination, in one embodiment, the innate immune system is activated within 24 hours of vaccination. The selection factor may be configured to minimize the controlling effect of monocytes on the NK cells. The selection factor is also configured to attract, proliferate, or mature antigen presenting cells or other prioritized cell types.

In general, the composition of the vaccine may be configured in a variety of ways. The composition may be configured to activate antigen presenting cells to initiate and maintain an immune response. The composition may also be configured to maintain the presentation of the antigenic target and selection factor for a defined time period to facilitate a memory immune response which outlasts the presentation of the antigenic target. The composition may also be configured to effect the adaptive immune system to minimize the upregulation of regulatory cells.

If the selection factor is released from the same device as the antigenic target and the immunogenic enhancing factor, the total amount of the selection factor administered per device may range from 0.5 micrograms to 10 micrograms per vaccination site, preferably between 2 and 4 micrograms.

In one embodiment, a method is provided for vaccinating a mammalian subject with a vaccination that activates the subject's innate immune system within 24 hours of the vaccination. Of course, the activation may be less than or greater than 24 hours depending upon the subject and the vaccination. The vaccination also provides a selection factor, cytokine such as GM-CSF, which minimizes the controlling effect of monocytes on the NK cells. The vaccination also presents an antigenic target and immunogenic enhancing factor or danger signal, which may be in the form of an adjuvant, to dendritic cells in a manner that activates the dendritic cells so that the dendritic cells present said antigen to immune systems so as to initiate and maintain an immune response.

In another embodiment, a method of vaccinating a mammalian immune system is provided which comprises a vaccination that activates the innate immune system within 24 hours of the vaccination including NK cells and T cells which will play an effector role in the innate immune system but also act upon the adaptive immune system to minimize the upregulation of the regulatory cells. The vaccination also provides a selection factor, a cytokine such as GM-CSF, which minimizes the controlling effect of monocytes on the NK cells. The vaccination also presents an antigenic target and the immunogenic enhancing factor or associated danger signal, which may be in the form of an adjuvant, to dendritic cells in a manner that activates the dendritic cells so that the DCs present said antigen to immune systems so as to initiate and maintain an immune response. In addition, the vaccination also maintains the presentation of the antigenic target and the immunogenic enhancing factor or danger signal for a period long enough to create a memory immune response which outlasts the presentation of the antigenic target by the vaccine.

A method of vaccinating a mammalian immune system is provided which comprises a vaccination that activates the innate immune system within 24 hours of the vaccination, including NK cells and T cells which will play an effector role in the innate immune system but also act upon the adaptive immune system to minimize the upregulation of the regulatory cells. The vaccination also provides a selection factor, a cytokine such as GM-CSF, which minimizes the controlling effect of monocytes on the NK cells. The vaccination also presents an antigenic target and the immunogenic enhancing factor or associated danger signal, which may be in the form of an adjuvant, to dendritic cells in a manner that activates the said dendritic cells—preferably plasmacytoid and myeloid dendritic cells so that the DCs present said antigen to immune systems, preferably the lymph nodes and or spleen, so as to initiate and maintain an immune response, preferably effected by T-cells. The vaccination also maintains the presentation of the antigenic target and the immunogenic enhancing factor or danger signal for a period long enough to create a memory immune response which outlasts the presentation of the antigenic target by the vaccine.

In another embodiment, a method of vaccinating a mammalian immune system is provided which comprises a vaccination that activates the innate immune system within 24 hours of the vaccination, including NK cells which will play an effector role in the innate immune system but also act upon the adaptive immune system to minimize the upregulation of the regulatory cells. The vaccination also provides a selection factor, a cytokine such as GM-CSF, which minimizes the controlling effect of monocytes on the NK cells and attracts and proliferates dendritic cells. The vaccination also presents an antigenic target and the immunogenic enhancing factor or associated danger signal, which may be in the form of an adjuvant, to dendritic cells in a manner that activates the dendritic cells so that the DCs present said antigenic target to immune systems so as to initiate and maintain an immune response.

In another embodiment, a method of increasing vaccine efficacy in a mammalian subject is provided. The method for vaccine efficacy includes implanting in the subject one or more delivery devices comprising an immunogenic enhancing factor and an antigenic target and administering to the subject a bolus containing at least a selection factor. Also, the vaccine efficacy may be increased by administering to the subject an injection or bolus composed of an immunogenic enhancing factor and a antigenic target at a location, site, or area proximal to the site, location, or area of the implanted one or more delivery devices. Alternatively, the bolus may be composed of an immunogenic factor and a tumor antigen and may be administered at a site proximal to the site of a delivery device comprising at least a selection factor.

Another method of increasing the vaccine efficacy is transdermally administering to the subject a composition comprising an immunogenic enhancing factor and an antigenic target at a location, site, or area proximal to the location, site, or area of the implanted one or more delivery device.

In another aspect, the invention comprises a method of programming dendritic cells in situ by introducing to a subject a matrix composition incorporating an immunogenic enhancing factor, selection factor, such as encapsulated GM-CSF, and an antigenic target and immediately thereafter up to 24 hours thereafter administering a bolus of the selection factor, wherein the bolus releases the selection factor upon introduction of the matrix and the matrix releases about 50-60% of the selection factor in a pulse between 1 and 7 days following introduction and releases the residual amount of selection factor incorporated into the matrix slowly over several weeks following introduction.

In both aspects, the matrix and bolus are preferentially administered locally at or near a site, or proximal to, having access to a lymph node that is not completely taken over by the tumor.

If the selection factor is released from a secondary delivery device proximal to or at a distance to the antigenic target and immunogenic enhancing factor, the total amount of the selection factor administered per device may vary from 0.5 micrograms to 20 micrograms per vaccination site preferably between 2 and 5 micrograms. With the dosage of the selection factor administered within the first 24 hours in vivo is between 1 and 90% of the total content preferably between 5 and 30%. The balance of the selection factor is released at a rate no greater than 15% per 24 hour period but preferably between 5 and 10% until all the selection factor is delivered to the patient.

In another embodiment, a method of vaccinating a mammalian immune system is disclosed. The vaccination causes an immediate foreign body or infection-like response and activates the innate immune system within 24 hours of the vaccination, including NK cells which will play an effector role in the innate immune system but also act upon the adaptive immune system to minimize the upregulation of the regulatory cell. The vaccination also provides a selection factor which minimizes the controlling effect of monocytes on the NK cells. The vaccination also presents an antigenic target and immunogenic enhancing factor, which may be in the form of an adjuvant, to dendritic cells in a manner that activates the said dendritic cells, preferably plasmacytoid and myeloid dendritic cells, so that the DCs present said antigenic target to immune systems, preferably the lymph nodes and or spleen, so as to initiate and maintain an immune response, preferably effected by T-cells. The vaccination also maintains the presentation of the antigenic target and immunogenic enhancing factor for a period long enough to create a memory t-cell response which outlasts the presentation of the antigenic target by the vaccine.

A vaccine of the invention is capable of presenting the antigenic target m immunogenic enhancing factor if used, on a surface whose micro and macro features, surface charge and long term pH preferentially favor close proximity and access by DCs.

A vaccine of the invention minimizes foreign body and fibrotic response (attraction of the wrong cell types) to the vaccination by controlling the surface of the vaccination vehicle or by incorporating an anti-inflammatory biomolecule into the vaccination vehicle.

In another embodiment, a three stage vaccine first releases selection factor; then provides available antigenic target and immunogenic enhancing factor, such as CpG, together once a sufficient number DCs are available at the vaccine site to activate the DCs (preferentially pDCs and mDCs) to prime the immune system and to activate the innate immune system; and then 10 to 40 days later makes antigenic target and immunogenic enhancing factor available to boost the immune system.

The foregoing has outlined, in general, the complete detailed description of the physical process, and or methods of application of the invention and is to serve as an aid to better understanding the intended application and use of the invention disclosed herein. In reference to such, there is to be a clear understanding the present invention is not limited to the method or detail of construction, fabrication, material, or application of use described and illustrated herein. Any other variation of fabrication, use, or application should be considered apparent as an alternative embodiment of the present invention.

In the foregoing specification, the invention has been described with reference to specific embodiments. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention.

Benefits, other advantages, and solutions to problems have been described above with regard to specific embodiments. However, the benefits, advantages, solutions to problems, and any element(s) that may cause any benefit, advantage, or solution to occur or become more pronounced are not to be construed as a critical, required, or essential feature, or element, of any or all the claims.

It would be appreciated by those skilled in the art that various changes and modifications can be made to the illustrated embodiments without departing from the spirit of the present invention. All such modifications and changes are intended to be covered by the appended claims.

What is claimed is:

1. A method of producing an immune response in situ of a subject comprising:
introducing one or more delivery devices having a morphology that prioritizes one or more prioritized cell types which interface with the one or more delivery devices.

2. The method of claim 1 wherein the prioritized cell types are selected from a group consisting of: NK cells, innate T cells, dendritic cells, other antigen presenting cells, and combinations thereof.

3. The method of claim 2, wherein the one or more delivery devices comprises a composition selected from a group consisting of: a selection factor, an antigenic target, and combinations thereof.

4. The method of claim 3, wherein the selection factor is configured to prioritize the prioritized cell types which interface with the one or more delivery devices.

5. The method of claim 4, wherein the selection factor is configured to actively attract, proliferate, or mature antigen presenting cells.

6. The method of claim 5, wherein the selection factor is a cytokine.

7. The method of claim 6, wherein the cytokine is GM-CSF.

8. The method of claim 3, further comprising:
an immunogenic enhancing factor.

9. The method of claim 8, wherein a first delivery device releases the selection factor and a second delivery device releases the antigenic target and the immunogenic enhancing factor.

10. The method of claim 8, wherein a second delivery device is located proximally to the first delivery device within 0 to 28 days after introducing the first delivery device.

11. The method of claim 10, wherein the second delivery device is located proximally to the first delivery device within 1 to 10 days after introducing the first delivery device.

12. The method of claim 8, wherein the one or more delivery devices comprising at least the antigenic target are located proximal to a tumor, and the one or more delivery devices continuously release the antigenic target to facilitate acquisition of the antigenic target by the prioritized cell types.

13. The method of claim 8, wherein the one or more delivery devices are configured to have a size and shape to facilitate antigen presenting cells acquisition of the antigenic target.

14. The method of claim 13, wherein the one or more delivery devices are configured to have surface pores whose size and shape facilitate the acquisition of the antigenic target from the one or more delivery devices by antigen presenting cells.

15. The method of claim 8, wherein the one or more delivery devices comprise one or more bioresorbable delivery devices configured to present the antigenic target on the surface of the one or more bioresorable delivery devices which are renewed when the one or more delivery bioresorable devices resorbs in vivo to provide fresh antigen for the antigen presenting cells.

16. The method of claim 8, wherein the one or more delivery devices comprises one or more bioresorbable delivery devices which locally release the antigenic target as the one or more bioresorbable delivery devices resorb to provide a locally increased concentration of the antigenic target for the antigen presenting cells to acquire.

17. The method of claim 16, wherein the one or more bioresorbable delivery devices release small particles to facilitate the antigen presenting cells acquisition of the antigenic target.

18. The method of claim 8, further comprising:
administering an injection comprising the immunogenic enhancing factor and the antigenic target at an area proximal to the location of the one or more delivery devices.

19. The method of claim 8, further comprising:
transdermally administering the immunogenic enhancing factor at an area proximal to the location of the one or more delivery devices.

20. The method of claim 8, wherein the composition is released in a bimodal manner with a first, initial burst of the composition being released within a first time period of 72 hours or less, and immediately following thereafter, a second, more gradual release of the composition continuing for a second time period.

21. The method of claim 20, wherein a remaining volume of the composition is released within the second time period and at a rate no greater than 1% of the volume per each 24 hour period.

22. The method of claim 21, wherein after three weeks, the remaining volume released per each 24 hour period varies between 0% and 35% of the remaining volume of composition.

23. The method of claim 8, further comprising:
administering a bolus comprising a selection factor; and
the one or more delivery devices comprising an immunogenic enhancing factor and antigenic target.

24. The method of claim 8, further comprising:
releasing the composition in a biologically appropriate dosage density and flux which does not attract unwanted cell types due to triggering an inflammatory or necrotic response.

25. The method of claim 24, wherein the biologically appropriate dosage and flux of the selection factor is between 0.1 and 600 nanograms/mm²/day.

26. The method of claim 24, wherein the biologically appropriate dosage and flux of the antigenic target is between 0.1 and 600 nanograms/mm²/day.

27. The method of claim 24, wherein the biologically appropriate dosage and flux of the immunogenic enhancing factor is between 0.1 and 600 nanograms/mm²/day.

28. A method of vaccinating to activate the innate immune system of a subject, comprising:
administering a vaccine comprising a composition selected from a group consisting of: a selection factor, an antigenic target, and combinations thereof.

29. The method of claim 28, wherein the composition further comprises:
an immunogenic enhancing factor.

29. The method of claim 29, wherein the innate immune system is activated within 24 hours of vaccination.

30. The method of claim 29, wherein selection factor is configured to minimize the controlling effect of monocytes on the NK cells.

31. The method of claim 29, wherein the composition is configured to activate antigen presenting cells to initiate and maintain an immune response.

32. The method of claim 29, wherein the composition is configured to maintain the presentation of the antigenic target and selection factor for a defined time period to facilitate a memory immune response which outlasts the presentation of the antigenic target.

33. The method of claim 29, wherein the composition is configured to effect the adaptive immune system to minimize the upregulation of regulatory cells.

34. The method of claim 29, wherein the selection factor is configured to attract, proliferate, or mature antigen presenting cells.
